(19) United States
(12) Patent Application Publication
(10) Pub. No.: US 2022/0153845 A1
(43) Pub. Date: May 19, 2022

(54) A METHOD FOR IMMUNOSUPPRESSION

(71) Applicant: BIOND BIOLOGICS LTD., Misgav (IL)

(72) Inventors: Motti HAKIM, Kibbutz Gazit (IL); Dror ALISHEKEVITZ, Kiryat Tivon (IL); Edna MEILIN, Kfar Veradim (IL); Ilana MANDEL, Manof (IL); Tehila BEN-MOSHE, Tel Aviv (IL); Yair SAPIR, Manof (IL); Avidor SHULMAN, Rakefet (IL)

(21) Appl. No.: 17/438,634

(22) PCT Filed: Mar. 12, 2020
(86) PCT No.: PCT/IL2020/050293

§ 371 Date: Sep. 13, 2021

Related U.S. Application Data
(60) Provisional application No. 62/818,336, filed on Mar. 14, 2019.
(60) Provisional application No. 62/942,240, filed on Dec. 2, 2019.

(51) Int. Cl. C07K 16/28; A61P 37/06

(52) U.S. Cl. CPC C07K 16/2818; A61P 37/06; C07K 2317/24; C07K 2317/51; C07K 2317/515; C07K 2317/565

(57) ABSTRACT

The present invention, in some embodiments thereof, is directed to a method for suppressing an immune response in a subject, including administering to the subject a therapeutically effective amount of an agent having specific binding affinity to a soluble immune receptor.

### CROSS REFERENCE TO RELATED APPLICATIONS

This application claims the benefit of priority of U.S. Provisional Patent Application No. 62/942,240, filed Dec. 2, 2019, and U.S. Provisional Patent Application No. 62/818,336, filed Mar. 14, 2019, and the contents of which are all incorporated herein by reference in their entirety.

### FIELD OF INVENTION

The present invention, in some embodiments thereof, is in the field of immunoregulation and immunotherapy.

### BACKGROUND OF THE INVENTION

It has been indicated that some co-stimulatory molecules have several physiological forms. Alongside membrane-bound forms, soluble forms have been described that are expressed in naive immune cells, increasing the complexity of T cell biology. The soluble form of CD28 (sCD28) has been ascribed to either specific proteolytic event or an alternatively spliced gene product. The splicing event results in a frame shift with the consequence of addition of two Glutamate residues after Glycine at position 137 before translational termination. The final product lacks the entire transmembrane and cytoplasmic regions and importantly is lacking the Cysteine residue, at position 141, that mediates the disulfide linkage of dimeric CD28 (Magistrelli et al., 1999). The biological function and counter-receptor binding of the monomeric CD28 soluble form was examined (Hebbar, 2004) and was shown to also inhibit T cell proliferation. In the case of dimeric sCD28, its function has not been shown though it has been suggested to have a regulatory role for T cell functionality by binding to B7 molecules (Sun, 2014). Contrary to the above, an elevation in the number of sCD28 molecules in the serum of patients with auto-immune disorders has been reported (Wong, C. K., Rheumatol, 2005; Hamzaoui, K., Clin Exp Rheumatol, 2005; Hebbar, M., Clin Exp Immunol, 2004; Sun, Z., Clin Immunol, 2014). In this respect, the clinical significance of sCD28 in autoimmune diseases and disorders is not known. A method of decreasing immune activation and treating an autoimmune disease by modulating sCD28 is therefore greatly needed.

### SUMMARY OF THE INVENTION

The present invention provides methods for suppressing an immune response in a subject, including administering to the subject a therapeutically effective amount of an agent having specific binding affinity to a soluble CD28.

According to a first aspect, there is provided a method for suppressing an immune response in a subject, comprising administering to the subject a therapeutically effective amount of an agent having specific binding affinity to soluble CD28 (sCD28), thereby suppressing an immune response in the subject.

According to some embodiments, the agent increases the serum level of the sCD28 in the subject.

According to some embodiments, the increase is at least a 20% increase as compared to the serum level without the administration.

According to some embodiments, the agent is not a CD28 agonist.

According to some embodiments, the agent is not a CD28 antagonist.

According to some embodiments, the agent binds sCD28 with at least a 2-fold greater binding affinity compared to the binding affinity of the agent to membrane CD28 (mCD28).

According to some embodiments, the agent does not bind mCD28.

According to some embodiments, the sCD28 is in serum.

According to some embodiments, the increasing the serum level of sCD28 comprises at least one of:

1) reducing sCD28 proteolysis;

2) reducing sCD28 degradation;

3) reducing sCD28 excretion;

4) increasing sCD28 half-life; and

5) any combination thereof.

According to some embodiments, the agent is an antibody or an antigen-binding portion thereof.

According to some embodiments, the antibody or antigen-binding portion thereof comprises an IgG2 or IgG4 backbone.

According to some embodiments, the antibody comprises three heavy chain CDRs (CDR-H) and three light chain CDRs (CDR-L), wherein:
CDR-H1 comprises the amino acid sequence set forth in SEQ ID NO: 1 (GYTLTNY), CDR-H2 comprises the amino acid sequence as set forth in SEQ ID NO: 2 (NTYTGK), CDR-H3 comprises the amino acid sequence as set forth in SEQ ID NO: 3 (GDANQQFAY), CDR-L1 comprises the amino acid sequence as set forth in SEQ ID NO: 4 (KASQDINSYLS), CDR-L2 comprises the amino acid sequence as set forth in SEQ ID NO: 5 (RANRLVD), and CDR-L3 comprises the amino acid sequence as set forth in SEQ ID NO: 6 (LQYDEFPPT);
   CDR-H1 comprises the amino acid sequence set forth in SEQ ID NO: 7 (GYTFTSY), CDR-H2 comprises the amino acid sequence as set forth in SEQ ID NO: 8 (YPGDGD), CDR-H3 comprises the amino acid sequence as set forth in SEQ ID NO: 9 (NYRYSSFGY), CDR-L1 comprises the amino acid sequence as set forth in SEQ ID NO: 10 (KSSQSLLNSGNQKNYLT), CDR-L2 comprises the amino acid sequence as set forth in SEQ ID NO: 11 (WASTRES), and CDR-L3 comprises the amino acid sequence as set forth in SEQ ID NO: 12 (QSDYSYPLT); or
   CDR-H1 comprises the amino acid sequence set forth in SEQ ID NO: 13 (GYTFTDY), CDR-H2 comprises the amino acid sequence as set forth in SEQ ID NO: 14 (NPNYDS), CDR-H3 comprises the amino acid sequence as set forth in SEQ ID NO: 15 (SSPYYDSNHFDY), CDR-L1 comprises the amino acid sequence as set forth in SEQ ID NO: 16 (SARSSINYMH), CDR-L2 comprises the amino acid sequence as set forth in SEQ ID NO: 17 (DTSKLAS), and CDR-L3 comprises the amino acid sequence as set forth in SEQ ID NO: 18 (HQRNSYPFT).

According to some embodiments, the antibody or an antigen-binding portion thereof comprises a heavy chain comprising the amino acid sequence of SEQ ID NO: 19, 20, 21, 22, 23, or 24.

According to some embodiments, the antibody or an antigen-binding portion thereof comprises a light chain comprising the amino acid sequence of SEQ ID NO: 25, 26, 27, 28, 29, or 30.

According to some embodiments, the antibody or an antigen-binding portion thereof is selected from the group consisting of: a Fv, Fab, F(ab′)2, scFv or a scFv2 fragment.

According to some embodiments, the antibody or an antigen-binding portion thereof is humanized.

According to some embodiments, the subject is a graft recipient.

According to some embodiments, the subject is afflicted with an autoimmune disease.

According to some embodiments, the autoimmune disease is a sCD28-positive autoimmune disease.

According to some embodiments, the autoimmune disease is selected from the group consisting of: lupus, rheumatoid arthritis, Crohn's disease, inflammatory bowel disease, Becht's disease, colitis, ulcerative colitis, diabetes, Graves' disease, and multiple sclerosis.

According to another aspect, there is provided a pharmaceutical composition comprising an agent having specific binding affinity to soluble CD28 (sCD28) for use in the treatment of an autoimmune disease.

According to another aspect, there is provided a pharmaceutical composition comprising an agent having specific binding affinity to soluble CD28 (sCD28) for use in increasing the serum level of sCD28.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1. is a graph showing that soluble CD28 diminishes lymphocytes clustering during stimulation with Staphylococcal enterotoxin B (SEB). Human peripheral blood mononuclear cells (PBMCs) were stimulated with SEB (1 ng/mL) without (black line with square marker) or in the presence of human IgG (dark gray line with triangle marker) or recombinant soluble human CD28 (gray line with circle marker). Cluster formation was monitored by using the IncuCyte® S3 Live-Cell System by taking cellular images at the indicated times.

FIGS. 2A-2C are vertical bar graphs showing that soluble CD28 inhibits effector cytokine secretion and promotes secretion of immune-suppressive cytokines in monocytes in an MLR setting. Isolated autologous monocytes and CD3 T cells were stimulated for 5 days with CMV peptide (0.5 μg/mL) without (black bars) or with increasing concentrations of recombinant human soluble CD28 (grey bars). Naïve samples without CMV stimulation are indicated by light grey bars. The concentration of human: IFNγ (2A), TGFβ (2B), and IL-10 (2C) in the supernatants were quantified with standardized sandwich ELISA (Biolegend).

FIG. 3 is a graph showing that an antibody targeting human soluble CD28 (sCD28) increases the serum exposure of the sCD28. The effect of anti-sCD28 (i.e., antibody #2) on the total sCD28 plasma concentration was evaluated in a co-injection in-vivo model in normal mice. Recombinant human sCD28 was intravenously injected as a single dose of 0.5 mg/kg without (grey line with circle marker) or with 5 mg/kg of antibody #2 (black line with square marker) and a time profile of total sCD28 plasma concentration is shown. Each data point represents the mean±s.d. (n=3 mice each).

FIGS. 4A-F: (4A-C) FACS histograms showing CD86 binding to cells expressing mCD28, after addition of CD86 alone (red lines) or addition of CD86 and (4A) CD28.2, (4B) Antibody #2, and (4C) mIgG control (green lines). Secondary antibody alone was added to show unstained cells (black lines). (4D-F) Bar charts showing Interferon gamma (IFNγ) secretion from (4D) T cells after treatment with 2 μg/mL anti-CD3, and CD28.2 (2.0 μg/mL) or antibody #2 in different dilutions (0.1-10 μg/mL, black bars), (4E) PBMCs after SEB stimulation and treatment with CD28.2 (2.0 μg/mL) or antibody #2 in different dilutions (0.1-10 μg/mL, black bars), and (4F) T cells after treatment with CD80-Fc with and without varying concentration of antibody #2.

### DETAILED DESCRIPTION OF THE INVENTION

The present invention, in some embodiments, provides methods of suppressing an immune response, and treating autoimmune disease comprising increasing sCD28 levels in a subject. The methods of the invention are based on the surprising finding that increased sCD28 levels reduced T cell clustering and the secretion of pro-inflammatory cytokines, e.g., interferon γ, and increased the secretion of anti-inflammatory cytokines, e.g., IL-10, and TGF-β. Further, it was unexpectedly found that an agent having specific binding affinity to sCD28 (i.e., an antibody) increased sCD28 serum levels in-vivo. Thus, an elevation of the serum levels of sCD28 in a subject's blood stream, could suppress a hyper immune response and, therefore, provide an effective anti-autoimmune therapy.

By a first aspect, there is provided a method for suppressing an immune response in a subject in need thereof, comprising administering to the subject a therapeutically effective amount of an agent having specific binding affinity to soluble CD28 (sCD28), thereby suppressing an immune response in the subject.

By another aspect, there is provided a method of suppressing an immune response in a subject in need thereof, the method comprising increasing the serum level of sCD28 in the subject.

In some embodiments, CD28 is mammalian CD28. In some embodiments the CD28 is human CD28. In some embodiments, the human CD28 comprises or consists of the amino acid sequence: MLRLLLALNLFPSIQVTGNKILVKQSPMLVAYDNAVNLSCKYSYNLFSREFRA SLHKGLDSAVEVCVVYGNYSQQLQVYSKTGFNCDGKLGNESVTFYLQNLYV NQTDIYFCKIEVMYPPPYLDNEKSNGTIIHVKGKHLCPSPLFPGPSKPFWVLVV VGGVLACYSLLVTVAFIIFWVRSKRSRLLHSDYMNMTPRRPGPTRKHYQPYAP PRDFAAYRS (SEQ ID NO: 31). In some embodiments, mature CD28 lacks a signal peptide and comprises or consists of the sequence:

(SEQ ID NO: 32)
NKILVKQSPMLVAYDNAVNLSCKYSYNLFSREFRASLHKGLDSAVEVCVV
YGNYSQQLQVYSKTGFNCDGKLGNESVTFYLQNLYVNQTDIYFCKIEVMY
PPPYLDNEKSNGTIIHVKGKHLCPSPLFPGPSKPFWVLVVVGGVLACYSL
LVTVAFIIFWVRSKRSRLLHSDYMNMTPRRPGPTRKHYQPYAPPRDFAAY
RS.

As used herein, “mCD28” refers to any CD28 which comprises a transmembrane domain and thus can be integrated in a membrane. In some embodiments, mCD28 is in a membrane. In some embodiments, mCD28 has passed from the ER, and through the Golgi into the plasma membrane of a cell. In some embodiments, mCD28 is in the plasma membrane of an immune cell. In some embodiments, mCD28 is in the plasma membrane of a T cell.

As used herein, “sCD28” refers to any CD28 fragment or variant that does not comprise a transmembrane domain and thus cannot be integrated in a membrane. In some embodiments, the CD28 transmembrane domain comprises the amino acid sequence FWVLVVVGGVLACYSLLVTVAFIIFWV (SEQ ID NO: 33). In some embodiments, sCD28 is not membrane bound. In some embodiments, sCD28 is in solution. In some embodiments, the sCD28 is CD28 in blood. In some embodiments, sCD28 is a cleaved form of full-length or mCD28. In some embodiments, sCD28 is dimeric. In some embodiments, sCD28 is monomeric. In some embodiments, sCD28 is a dimer cleavage produce of mCD28. In some embodiments, sCD28 does not comprise the entire extracellular domain of mCD28. In some embodiments, sCD28 is CD28 in a bodily fluid. In some embodiments, sCD28 lacks exon 3 of CD28. In some embodiments, sCD28 is a cleavage product from mCD28. In some embodiments, sCD28 is truncated CD28. In some embodiments, sCD28 lacks the cytoplasmic domain of full-length CD28. In some embodiments, sCD28 comprises the amino acid sequence: MLRLLLALNLFPSIQVTGNKILVKQSPMLVAYDNAVNLSCKYSYNLFSREFRA SLHKGLDSAVEVCVVYGNYSQQLQVYSKTGFNCDGKLGNESVTFYLQNLYV NQTDIYFCKIEVMYPPPYLDNEKSNGTIIHVKGEE (SEQ ID NO: 34). In some embodiments, sCD28 consists of SEQ ID NO: 34. In some embodiments, sCD28 lacks the signal peptide and comprises the sequence: NKILVKQSPMLVAYDNAVNLSCKYSYNLFSREFRASLHKGLDSAVEVCVVYG NYSQQLQVYSKTGFNCDGKLGNESVTFYLQNLYVNQTDIYFCKIEVMYPPPYL DNEKSNGTIIHVKGEE (SEQ ID NO: 35). In some embodiments, sCD28 consists of SEQ ID NO: 35. In some embodiments, sCD28 comprises the amino acid sequence: MLRLLLALNLFPSIQVTGNKILVKQSPMLVAYDNAVNLSCKYSYNLFSREFRA SLHKGLDSAVEVCVVYGNYSQQLQVYSKTGFNCDGKLGNESVTFYLQNLYV NQTDIYFCKIEVMYPPPYLDNEKSNGTIIHVKGKHLCPSP (SEQ ID NO: 39). In some embodiments, sCD28 consists of the amino acid sequence of SEQ ID NO: 39. In some embodiments, sCD28 lacks the signal peptide and comprises the sequence: NKILVKQSPMLVAYDNAVNLSCKYSYNLFSREFRASLHKGLDSAVEVCVVYG NYSQQLQVYSKTGFNCDGKLGNESVTFYLQNLYVNQTDIYFCKIEVMYPPPYL DNEKSNGTIIHVKGKHLCPSP (SEQ ID NO: 40). In some embodiments, sCD28 consists of the amino acid sequence of SEQ ID NO: 40.

In some embodiments, sCD28 has a variable C-terminus. In some embodiments, sCD28 terminates at a cleavage site within the membrane proximal region of CD28. In some embodiments, sCD28 terminates at a cleavage site within the stalk region of CD28. In some embodiments, the stalk region comprises the sequence GKHLCPSPLFPGPSKP (SEQ ID NO: 36). In some embodiments, the stalk region comprises or consists of the sequence HVKGKHLCPSPLFPGPSKP (SEQ ID NO: 37). In some embodiments, sCD28 terminates at leucine 145 of SEQ ID NO: 31. In some embodiments, sCD28 terminates at leucine 127 of SEQ ID NO: 32.

In some embodiments, the cleavage site is before a Leucine. In some embodiments, the cleavage site is before a Valine. In some embodiments, the cleavage site is before an aromatic amino acid. In some embodiments, the cleavage site is before a Leucine, Valine and/or aromatic amino acid. In some embodiments, the aromatic amino acid is selected from Phenylalanine, Tryptophan, Tyrosine and Histidine. In some embodiments, the cleavage site is before any one of Histidine 134, Valine 135, Histidine 139, Leucine 140, Leucine 145, and Phenylalanine 146 of SEQ ID NO: 21. In some embodiments, the cleavage site is before Histidine 134, Valine 135, Histidine 139, Leucine 140, Leucine 145, or phenylalanine 146 of SEQ ID NO: 31. Each possibility represents a separate embodiment of the invention. In some embodiments, the cleavage site is before Leucine 145 of SEQ ID NO: 31. In some embodiments, the cleavage site is before Leucine 127 of SEQ ID NO: 32.

In some embodiments, sCD28 levels are sCD28 serum levels. In some embodiments, sCD28 levels are sCD28 blood levels. In some embodiments, sCD28 levels are systemic levels. In some embodiments, sCD28 levels are local levels. In some embodiments, the local levels are at a site of immune response.

In some embodiments, the agent is not a CD28 antagonist. In some embodiments, the agent is not a CD28 agonist. In some embodiments, the agent is a CD28 agonist. In some embodiments, the agent is neither a CD28 agonist nor antagonist. In some embodiments, the agent does not directly affect mCD28 signaling. In some embodiments, the agent is not a CD28 direct agonist. In some embodiments, the agent is not a CD28 direct antagonist. In some embodiments, the agent has an indirect antagonist effect by increasing sCD28 levels. In some embodiments, the agent does not bind mCD28.

The term “agonist” generally refers to a molecule, compound or agent that binds to a receptor and activates, fully or partially, the receptor. In some embodiments, the agonist binds at the same site as the natural ligand. In some embodiments, the agonist binds at an allosteric site different from the binding site of the natural ligand. The term “antagonist” generally refers to a molecule, compound or agent that binds to a receptor at the same site as an agonist or another site, does not activate the receptor and does one or more of the following: interferes with or blocks activation of the receptor by a natural ligand, and interferes with or blocks activation of the receptor by a receptor agonist.

As used herein, a “direct agonist/antagonist” refers to a molecule that binds to a receptor (mCD28) and by binding increases/decreases signaling by that molecule. In the case of mCD28 a direct agonist would bind mCD28 and by binding increase mCD28 signaling in the cell. In some embodiments, the agonist increases T cell activation. In some embodiments, the agonist increases T cell proliferation. In some embodiments, the agonist increases pro-inflammatory cytokine secretion. Pro-inflammatory cytokines are well known in the art and are known to be secreted by activated T cells. Examples of pro-inflammatory cytokines include, but are not limited to, TNFα, IFNγ, IL-1B, and IL-6. In some embodiments, the pro-inflammatory cytokine is IFNγ. In the case of mCD28, a direct antagonist would bind mCD28 and by binding decrease mCD28 signaling in the cell. In some embodiments, the antagonist decreases T cell activation, decreases T cell proliferation and/or decreases pro-inflammatory cytokine secretion. A molecule that effects a receptor's signaling by contacting its ligand, contacting an inhibitor, contacting a co-receptor or contacting any molecule other than the receptor in question in order to modify receptor signaling is not considered a direct agonist/antagonist. In some embodiments, the agent of the invention contacts sCD28 in serum and thereby allows for decreased signaling through mCD28 on cells. Though the result is decreased mCD28 signaling the antibody is not a mCD28 antagonist or direct antagonist as it does not bind to mCD28.

In some embodiments, the agent does not bind the ligand binding domain of CD28. In some embodiments, the agent does not bind the ligand binding domain of sCD28. In some embodiments, the agent does not bind the ligand binding domain of mCD28. In some embodiments, the agent does not obscure or block access to the ligand binding domain. In some embodiments, the agent does not bind, obscure or block access to the IgV domain of CD28, sCD28 or mCD28. Each possibility represents a separate embodiment of the invention. In some embodiments, the IgV domain is the ligand binding domain. In some embodiments, the ligand binding domain comprises amino acids 28-137 of SEQ ID NO: 1. In some embodiments, the ligand binding domain comprises or consists of the amino acid sequence MLVAYDNAVNLSCKYSYNLFSREFRASLHKGLDSAVEVCVVYGNYSQQLQV YSKTGFNCDGKLGNESVTFYLQNLYVNQTDIYFCKIEVMYPPPYLDNEKSNGT IIHVKG (SEQ ID NO: 38). In some embodiments, the agent does not inhibit binding of CD28 to a ligand. In some embodiments, the CD28 ligand is CD80, CD86, ICOSL or a combination thereof. In some embodiments, the CD28 ligand is CD86. In some embodiments, the CD28 ligand is CD80. In some embodiments, CD86 is CD86-Fc. In some embodiments, CD80 is CD80-Fc. In some embodiments, the CD28 ligand is ICOSL.

In some embodiments, the agent increases the serum level of sCD28 in the subject. In some embodiments, increasing the serum level comprising increasing the half-life of the sCD28. In some embodiments, the agent increases the half-life of sCD28. In some embodiments, increasing the serum level comprises reducing sCD28 proteolysis, degradation, excretion, or any combination thereof. In some embodiments, increasing the serum level comprises reducing sCD28 proteolysis. In some embodiments, increasing the serum level comprises reducing sCD28 degradation. In some embodiments, increasing the serum level comprises reducing sCD28 excretion. In some embodiments, the agent reduces sCD28 proteolysis, degradation, excretion, or any combination thereof.

As used herein, “proteolysis” refers to cleavage, breakdown, or both, of a protein into smaller fragments, e.g., peptides, polypeptides or single amino acids, primarily by a protease.

As used herein, “degradation of a protein” encompasses any type of a protein breakdown, e.g., enzymatic, chemical, or physical. In one embodiment, protein degradation takes place intracellularly. In one embodiment, protein degradation takes place extracellularly. In one embodiment, protein degradation takes place intracellularly and extracellularly. In one embodiment, protein degradation takes place in the proteasome complex.

As used herein, “excretion” refers to any process by which a protein is removed from the body of an organism.

In some embodiments, reduction of sCD28 proteolysis, degradation, excretion, or any combination thereof, is a reduction of at least 5, 10, 15, 20, 25, 30, 35, 40, 45, 50, 55, 60, 65, 70, 75, 80, 85, 90, 95, 97, 99 or 100% reduction of sCD28 proteolysis, degradation, excretion, or any combination thereof, or any value and range therebetween. Each possibility represents a separate embodiment of the invention. In some embodiments, reducing sCD28 proteolysis, degradation, excretion, or any combination thereof, maintains levels of sCD28 in serum. In some embodiments, reducing sCD28 proteolysis, degradation, excretion, or any combination thereof, increases levels of sCD28 in serum. In some embodiments, reducing sCD28 proteolysis, degradation, excretion, or any combination thereof, maintains levels of sCD28 adequate for immune suppression. In some embodiments, reducing sCD28 proteolysis, degradation, excretion, or any combination thereof, induces levels of sCD28 adequate for immune suppression.

In some embodiments, the agent reduces T cell activation. In some embodiments, the agent reduces T cell proliferation. In some embodiments, the agent reduces T cell clustering. In some embodiments, the agent increases anti-inflammatory cytokine secretion. Anti-inflammatory cytokines are well known in the art. Non-limiting examples of anti-inflammatory cytokines include, but are not limited to, IL-10, and TGFβ. In some embodiments, the agent decreases pro-inflammatory cytokine secretion. In some embodiments, the pro-inflammatory cytokine is IFNγ.

In some embodiments, the agent does not modulate CD28 function and/or signaling. In some embodiments, the agent increases or maintains sCD28 levels. In some embodiments, the agent leads to or facilitates stabilization of sCD28. In some embodiments, the signaling is sCD28-mediated immune suppression. In some embodiments, the signaling is CD28-mediated immune response. In some embodiments, the agent increases or promotes immune suppression.

Thus, an agent that stabilizes sCD28, and is also not a direct antagonist of mCD28 signaling, could therefore suppress immune cells, e.g., T-lymphocytes and secretion of pro-inflammatory cytokines therefrom, and potentially suppress an immune response.

In some embodiments the agent does not induce antibody dependent cell-mediated cytotoxicity (ADCC). In some embodiments, the agent does not induce complement-dependent cytotoxicity (CDC). In some embodiments, the agent does not induce ADCC and/or CDC. In some embodiments, the agent is an antibody and comprises an IgG2 or IgG4 domain. In some embodiments, the antibody comprises an IgG2 domain. In some embodiments, IgG2 is selected from IgG2a and IgG2b. In some embodiments, IgG2 is IgG2b. In some embodiments, the antibody comprises an IgG4 domain. In some embodiments, the antibody comprises an IgG1 or IgG3 mutated to reduce cell death mediated by binding of the antibody. In some embodiments, the mutation mutates a Fc receptor binding domain. In some embodiments, a Fc domain of the antibody is engineered or mutated to decrease CDC, ADCC or both. Fc engineering is well known in the art, and any mutation or amino acid change that is known to decrease antibody mediated cell killing may be used.

In some embodiments, the agent is an antibody or an antigen binding fragment thereof. In some embodiments, the agent is a small molecule. In some embodiments, the agent is a nucleic acid molecule. In some embodiments, the agent is a synthetic peptide. In some embodiments, the agent is a synthetic binding protein. In some embodiments, the synthetic peptide is based on a non-antibody scaffold. In some embodiments, the agent is an antibody mimetic. In some embodiments, the antibody mimetic has a molar mass of less than 100, 90, 80, 70, 60, 50, 40, 30 or 20 kDa. Each possibility represents a separate embodiment of the invention. In some embodiments, the agent is a nucleic acid aptamer. In some embodiments, the aptamer is DNA. In some embodiments, the aptamer is RNA. In some embodiments, the aptamer is DNA or RNA. Examples of antibody mimetics include, but are not limited to, affilins, affimers, affitins, alphabodies, anticalins, avimers, DARPins, fynomers, Kunitz domain peptides, monobodies, and nanoCLAMPS. In some embodiments, the antibody mimetic is a DARPin. In some embodiments, the agent is a non-antibody protein.

In some embodiments, the agent targets sCD28. In some embodiments, the target of the agent is sCD28, and/or dimeric sCD28. In some embodiments, the target of the agent is sCD28, and/or monomeric sCD28. In some embodiments, the target of the agent is sCD28, monomeric sCD28, and/or dimeric sCD28. In some embodiments, the sCD28 is monomeric. In some embodiments, the sCD28 is dimeric. In some embodiments, the sCD28 is monomeric or dimeric. In some embodiments, the agent is an anti-sCD28 antibody. An “anti-sCD28 antibody”, “an antibody which recognizes sCD28”, or “an antibody against sCD28” is an antibody that binds sCD28, with sufficient affinity and specificity. In some embodiments, the agent has increased binding to sCD28. In some embodiments, the agent has increased binding to sCD28 as compared to mCD28. In some embodiments, the agent has specific binding affinity for sCD28.

As used herein, the terms “increased binding affinity” and “greater binding affinity” are interchangeable. In some embodiments, the agent has a greater binding affinity to sCD28 as compared to the mCD28. In one embodiment, greater affinity as used herein is by at least 10%. In one embodiment, greater affinity as used herein is by at least 30%. In one embodiment, greater affinity as used herein is by at least 50%. In one embodiment, greater affinity as used herein is by at least 75%. In one embodiment, greater affinity as used herein is by at least 100%. In one embodiment, greater affinity as used herein is by at least 150%. In one embodiment, greater affinity as used herein is by at least 250%. In one embodiment, greater affinity as used herein is by at least 500%. In one embodiment, greater affinity as used herein is by at least 1,000%. In one embodiment, greater affinity as used herein is by at least 1.5-fold. In one embodiment, greater affinity as used herein is by at least 2-fold. In one embodiment, greater affinity as used herein is by at least 5-fold. In one embodiment, greater affinity as used herein is by at least 10-fold. In one embodiment, greater affinity as used herein is by at least 50-fold. In one embodiment, greater affinity as used herein is by at least 100-fold. In one embodiment, greater affinity as used herein is by at least 500-fold. In one embodiment, greater affinity as used herein is by at least 1,000-fold.

In some embodiments, the agent is a single domain antibody. In some embodiments, the antibody lacks a Fc domain. In some embodiments, the agent is an antigen binding domain that lacks an Fc domain. In some embodiments, the agent is a single-domain antibody. In some embodiments, the agent is a camelid antibody, shark antibody or nanobody. In some embodiments, the antibody or fragment is fused to another protein or fragment of a protein. In some embodiments, the second protein or fragment increases the half-life of the agent, the levels in the serum. In some embodiments, the agent's half-life extending protein is human serum albumin. In some embodiments, the agent is modified by a chemical that produces a modification that enhances the agent's half-life. In some embodiments, the modification is PEGylation and the chemical is polyethylene glycol. A skilled artisan will appreciate that any half-life extending protein or chemical agent, or modification known in the art may be used.

In some embodiments, according to the method of the present invention, an agent is an antibody or an antigen-binding portion thereof.

In some embodiments, the antibody is “Antibody #1”. In some embodiments, Antibody #1 comprises three heavy chain CDRs (CDR-H) and three light chain CDRs (CDR-L), wherein: CDR-H1 comprises the amino acid sequence set forth in SEQ ID NO: 1 (GYTLTNY), CDR-H2 comprises the amino acid sequence as set forth in SEQ ID NO: 2 (NTYTGK), CDR-H3 comprises the amino acid sequence as set forth in SEQ ID NO: 3 (GDANQQFAY), CDR-L1 comprises the amino acid sequence as set forth in SEQ ID NO: 4 (KASQDINSYLS), CDR-L2 comprises the amino acid sequence as set forth in SEQ ID NO: 5 (RANRLVD), and CDR-L3 comprises the amino acid sequence as set forth in SEQ ID NO: 6 (LQYDEFPPT).

In some embodiments, the antibody is “Antibody #2”. In some embodiments, Antibody #2 comprises three CDR-H and three CDR-L, wherein: CDR-H1 comprises the amino acid sequence set forth in SEQ ID NO: 7 (GYTFTSY), CDR-H2 comprises the amino acid sequence as set forth in SEQ ID NO: 8 (YPGDGD), CDR-H3 comprises the amino acid sequence as set forth in SEQ ID NO: 9 (NYRYSSFGY), CDR-L1 comprises the amino acid sequence as set forth in SEQ ID NO: 10 (KSSQSLLNSGNQKNYLT), CDR-L2 comprises the amino acid sequence as set forth in SEQ ID NO: 11 (WASTRES), and CDR-L3 comprises the amino acid sequence as set forth in SEQ ID NO: 12 (QSDYSYPLT).

In some embodiments, the antibody is “Antibody #3”. In some embodiments, Antibody #3 comprises three CDR-H and three CDR-L, wherein: CDR-H1 comprises the amino acid sequence set forth in SEQ ID NO: 13 (GYTFTDY), CDR-H2 comprises the amino acid sequence as set forth in SEQ ID NO: 14 (NPNYDS), CDR-H3 comprises the amino acid sequence as set forth in SEQ ID NO: 15 (SSPYYDSNHFDY), CDR-L1 comprises the amino acid sequence as set forth in SEQ ID NO: 16 (SARSSINYMH), CDR-L2 comprises the amino acid sequence as set forth in SEQ ID NO: 17 (DTSKLAS), and CDR-L3 comprises the amino acid sequence as set forth in SEQ ID NO: 18 (HQRNSYPFT).

In some embodiments, the antibody or antigen-binding fragment thereof comprises a heavy chain comprising the amino acid sequence QIQLVQSGPELKKPGETVKISCKASGYTLTNYGMNWVKQAPGKGLKWMGWI NTYTGKPTYVDDFKGRFAFSLETSASTAYLQINNLKNEDTATYFCARGDANQ QFAYWGQGTLVTVS (SEQ ID NO: 19). In some embodiments, the variable region of the heavy chain comprises and/or consists of SEQ ID NO: 19. In some embodiments, the antibody or antigen-binding fragment thereof comprises a heavy chain comprising the amino acid sequence QIQLVQSGPELKKPGETVKISCKASGYTLTNYGMNWVKQAPGKGLKWMGWI NTYTGKPTYVDDFKGRFAFSLETSASTAYLQINNLKNEDTATYFCARGDANQ QFAYWGQGTLVTVSAAKTTPPSVYPLAPGSAAQTNSMVTLGCLVKGYFPEPV TVTWNSGSLSSGVHTFPAVLQSDLYTLSSSVTVPSSTWPSETVTCNVAHPASST KVDKKIVPRDCGCKPCICTVPEVSSVFIFPPKPKDVLTITLTPKVTCVVVDISKD DPEVQFSWFVDDVEVHTAQTQPREEQFNSTERSVSELPIMHQDWLNGKEFKC RVNSAAFPAPIEKTISKTKGRPKAPQVYTIPPPKEQMAKDKVSLTCMITDFFPED ITVEWQWNGQPAENYKNTQPIMDTDGSYFVYSKLNVQKSNWEAGNTFTCSV LHEGLHNHHTEKSLSHSPGK (SEQ ID NO: 20). In some embodiments, the heavy chain consists of SEQ ID NO: 20. Antibody #1, as used herein has a heavy chain consisting of SEQ ID NO: 20 and the CDRs of this heavy chain are SEQ ID Nos.: 1-3.

In some embodiments, the antibody or antigen-binding fragment thereof comprises a light chain comprising the amino acid sequence DIKMTQSPSSMYASLGERVTITCKASQDINSYLSWFQQKPGKSPKTLIYRANRL VDGVPSRFSGSGSGQDYSLTISSLEYDDMGIYYCLQYDEFPPTFGAGTKLELK (SEQ ID NO: 25). In some embodiments, the variable region of the light chain comprises and/or consists of SEQ ID NO: 25. In some embodiments, the antibody or antigen-binding fragment thereof comprises a light chain comprising the amino acid sequence DIKMTQSPSSMYASLGERVTITCKASQDINSYLSWFQQKPGKSPKTLIYRANRL VDGVPSRFSGSGSGQDYSLTISSLEYDDMGIYYCLQYDEFPPTFGAGTKLELKR ADAAPTVSIFPPSSEQLTSGGASVVCFLNNFYPKDINVKWKIDGSERQNGVLNS WTDQDSKDSTYSMSSTLTLTKDEYERHNSYTCEATHKTSTSPIVKSFNRNEC (SEQ ID NO: 26). In some embodiments, the light chain consists of SEQ ID NO: 26. Antibody #1, as used herein has a light chain consisting of SEQ ID NO: 26 and the CDRs of this light chain are SEQ ID Nos.: 4-6.

In some embodiments, the antibody or antigen-binding fragment thereof comprises a heavy chain comprising the amino acid sequence QVQLQQSGAELARPGASVKLSCKASGYTFTSYWMQWIKKRPGQGLEWIGAIY PGDGDTRYTQKFKGKATLTADKSSTTAYMQLSSLASEDSAVYFCARNYRYSS FGYWGQGTLVTVSA (SEQ ID NO: 21). In some embodiments, the variable region of the heavy chain comprises and/or consists of SEQ ID NO: 21. In some embodiments, the antibody or antigen-binding fragment thereof comprises a heavy chain comprising the amino acid sequence QVQLQQSGAELARPGASVKLSCKASGYTFTSYWMQWIKKRPGQGLEWIGAIY PGDGDTRYTQKFKGKATLTADKSSTTAYMQLSSLASEDSAVYFCARNYRYSS FGYWGQGTLVTVSAAKTTPPSVYPLAPGCGDTTGSSVTLGCLVKGYFPESVTV TWNSGSLSSSVHTFPALLQSGLYTMSSSVTVPSSTWPSQTVTCSVAHPASSTTV DKKLEPSGPISTINPCPPCKECHKCPAPNLEGGPSVFIFPPNIKDVLMISLTPKVT CVVVDVSEDDPDVQISWFVNNVEVHTAQTQTHREDYNSTIRVVSTLPIQHQD WMSGKEFKCKVNNKDLPSPIERTISKIKGLVRAPQVYILPPPAEQLSRKDVSLT CLVVGFNPGDISVEWTSNGHTEENYKDTAPVLDSDGSYFIYSKLNMKTSKWE KTDSFSCNVRHEGLKNYYLKKTISRSPGK (SEQ ID NO: 22). In some embodiments, the heavy chain consists of SEQ ID NO: 22. Antibody #2 as used herein, has a light chain consisting of SEQ ID NO: 22 and the CDRs of this heavy chain are SEQ ID Nos.: 7-9.

In some embodiments, the antibody or antigen-binding fragment thereof comprises a light chain comprising the amino acid sequence DIVMTQSPSSLTVTAGEKVTLSCKSSQSLLNSGNQKNYLTWYQQKPGQPPQLL IYWASTRESGVPDRFTGSGSGTDFTLTISSVQAEDLAVYYCQSDYSYPLTFGAG TKLELK (SEQ ID NO: 27). In some embodiments, the variable region of the light chain comprises and/or consists of SEQ ID NO: 27. In some embodiments, the antibody or antigen-binding fragment thereof comprises a light chain comprising the amino acid sequence DIVMTQSPSSLTVTAGEKVTLSCKSSQSLLNSGNQKNYLTWYQQKPGQPPQLL IYWASTRESGVPDRFTGSGSGTDFTLTISSVQAEDLAVYYCQSDYSYPLTFGAG TKLELKRADAAPTVSIFPPSSEQLTSGGASVVCFLNNFYPKDINVKWKIDGSER QNGVLNSWTDQDSKDSTYSMSSTLTLTKDEYERHNSYTCEATHKTSTSPIVKS FNRNEC (SEQ ID NO: 28). In some embodiments, the light chain consists of SEQ ID NO: 28. In some embodiments, the light chain consists of SEQ ID NO: 28. Antibody #2 as used herein, has a light chain consisting of SEQ ID NO: 28 and the CDRs of this light chain are SEQ ID Nos.: 10-12.

In some embodiments, the antibody or antigen-binding fragment thereof comprises a heavy chain comprising the amino acid sequence EVQLQQFGAELVKPGASVKISCKASGYTFTDYNMDWVKQSHGKSLEWIGDIN PNYDSTAYNQKFMGKATLTVDKSSNTAYMELRSLTSEDTAVYYCARSSPYYD SNHFDYWGQGTSLTVSS (SEQ ID NO: 23). In some embodiments, the variable region of the heavy chain comprises and/or consists of SEQ ID NO: 23. In some embodiments, the antibody or antigen-binding fragment thereof comprises a heavy chain comprising the amino acid sequence EVQLQQFGAELVKPGASVKISCKASGYTFTDYNMDWVKQSHGKSLEWIGDIN PNYDSTAYNQKFMGKATLTVDKSSNTAYMELRSLTSEDTAVYYCARSSPYYD SNHFDYWGQGTSLTVSSAKTTPPSVYPLAPGSAAQTNSMVTLGCLVKGYFPEP VTVTWNSGSLSSGVHTFPAVLQSDLYTLSSSVTVPSSTWPSETVTCNVAHPASS TKVDKKIVPRDCGCKPCICTVPEVSSVFIFPPKPKDVLTITLTPKVTCVVVDISK DDPEVQFSWFVDDVEVHTAQTQPREEQFNSTFRSVSELPIMHQDWLNGKEFK CRVNSAAFPAPIEKTISKTKGRPKAPQVYTIPPPKEQMAKDKVSLTCMITDFFPE DITVEWQWNGQPAENYKNTQPIMDTDGSYFVYSKLNVQKSNWEAGNTFTCS VLHEGLHNHHTEKSLSHSPGK (SEQ ID NO: 24). In some embodiments, the heavy chain consists of SEQ ID NO: 24. Antibody #3 as used herein, has a heavy chain consisting of SEQ ID NO: 24 and the CDRs of this heavy chain are SEQ ID Nos.: 13-15.

In some embodiments, the antibody or antigen-binding fragment thereof comprises a light chain comprising the amino acid sequence QIVLTQSPAIMSASPGEKVTMTCSARSSINYMIHWFQQKPGTSPKRWIYDTSKL ASGVPARFSGSGSGTSYSLTISNMEAEDAATYYCHQRNSYPFTFGSGTKLEIK (SEQ ID NO: 29). In some embodiments, the variable region of the light chain comprises and/or consists of SEQ ID NO: 29. In some embodiments, the antibody or antigen-binding fragment thereof comprises a light chain comprising the amino acid sequence QIVLTQSPAIMSASPGEKVTMTCSARSSINYMIHWFQQKPGTSPKRWIYDTSKL ASGVPARFSGSGSGTSYSLTISNMEAEDAATYYCHQRNSYPFTFGSGTKLEIKR ADAAPTVSIFPPSSEQLTSGGASVVCFLNNFYPKDINVKWKIDGSERQNGVLNS WTDQDSKDSTYSMSSTLTLTKDEYERHNSYTCEATHKTSTSPIVKSFNRNEC (SEQ ID NO: 30). In some embodiments, the light chain consists of SEQ ID NO: 30. Antibody #3 as used herein, has a light chain consisting of SEQ ID NO: 30 and the CDRs of this light chain are SEQ ID NOs: 16-18.

The term “antibody” (also referred to as an “immunoglobulin”) is used in the broadest sense and specifically encompasses monoclonal antibodies and antibody fragments so long as they exhibit the desired biological activity. In certain embodiments, the use of a chimeric antibody or a humanized antibody is also encompassed by the method of the invention. In some embodiments, the antibody is a humanized antibody comprising the aforementioned CDRs.

Generally, an antibody refers to a polypeptide or group of polypeptides that include at least one binding domain that is formed from the folding of polypeptide chains having three-dimensional binding spaces with internal surface shapes and charge distributions complementary to the features of an antigenic determinant of an antigen. An antibody typically has a tetrameric form, comprising two identical pairs of polypeptide chains, each pair having one “light” and one “heavy” chain. The variable regions of each light/heavy chain pair form an antibody binding site. An antibody may be oligoclonal, polyclonal, monoclonal, chimeric, camelid, CDR-grafted, multi-specific, bi-specific, catalytic, humanized, fully human, anti-idiotypic and antibodies that can be labeled in soluble or bound form as well as fragments, including epitope-binding fragments, variants or derivatives thereof, either alone or in combination with other amino acid sequences. An antibody may be from any species. The term antibody also includes binding fragments, including, but not limited to Fv, Fab, Fab′, F(ab′)2 single stranded antibody (svFc), dimeric variable region (Diabody) and disulfide-linked variable region (dsFv). In particular, antibodies include immunoglobulin molecules and immunologically active fragments of immunoglobulin molecules, i.e., molecules that contain an antigen binding site. Antibody fragments may or may not be fused to another immunoglobulin domain including but not limited to, an Fc region or fragment thereof. The skilled artisan will further appreciate that other fusion products may be generated including but not limited to, scFv-Fc fusions, variable region (e.g., VL and VH)-Fc fusions and scFv-scFv-Fc fusions.

Immunoglobulin molecules can be of any type (e.g., IgG, IgE, IgM, IgD, IgA, and IgY), class (e.g., IgG1, IgG2, IgG3, IgG4, IgA1 and IgA2) or subclass. In some embodiments, the antibody comprises IgG2 or IgG4. In some embodiments, the antibody comprises IgG2. In some embodiments, the antibody comprises IgG4.

The basic unit of the naturally occurring antibody structure is a heterotetrameric glycoprotein complex of about 150,000 Daltons, composed of two identical light (L) chains and two identical heavy (H) chains, linked together by both noncovalent associations and by disulfide bonds. Each heavy and light chain also has regularly spaced intra-chain disulfide bridges. Five human antibody classes (IgG, IgA, IgM, IgD and IgE) exist, and within these classes, various subclasses, are recognized based on structural differences, such as the number of immunoglobulin units in a single antibody molecule, the disulfide bridge structure of the individual units, and differences in chain length and sequence. The class and subclass of an antibody is its isotype.

The amino terminal regions of the heavy and light chains are more diverse in sequence than the carboxy terminal regions, and hence are termed the variable domains. This part of the antibody structure confers the antigen-binding specificity of the antibody. A heavy variable (VH) domain and a light variable (VL) domain together form a single antigen-binding site, thus, the basic immunoglobulin unit has two antigen-binding sites. Particular amino acid residues are believed to form an interface between the light and heavy chain variable domains (Chothia et al., J. Mol. Biol. 186, 651-63 (1985); Novotny and Haber, (1985) Proc. Natl. Acad. Sci. USA 82 4592-4596).

The carboxy terminal portion of the heavy and light chains form the constant domains i.e. CH1, CH2, CH3, CL. While there is much less diversity in these domains, there are differences from one animal species to another, and further, within the same individual there are several different isotypes of antibody, each having a different function.

The term “framework region” or “FR” refers to the amino acid residues in the variable domain of an antibody, which are other than the hypervariable region amino acid residues as herein defined. The term “hypervariable region” as used herein refers to the amino acid residues in the variable domain of an antibody, which are responsible for antigen binding. The hypervariable region comprises amino acid residues from a “complementarity determining region” or “CDR”. The CDRs are primarily responsible for binding to an epitope of an antigen. The extent of FRs and CDRs has been precisely defined (see, Kabat et al.).

Immunoglobulin variable domains can also be analyzed using the IMGT information system (www://imgt. cines.fr/) (IMGT®/V-Quest) to identify variable region segments, including CDRs. See, e.g., Brochet, X. et al, Nucl. Acids Res. J6:W503-508 (2008).

Chothia et al. also defined a numbering system for variable domain sequences that is applicable to any antibody. One of ordinary skill in the art can unambiguously assign this system of “Chothia numbering” to any variable domain sequence, without reliance on any experimental data beyond the sequence itself. As used herein, “Chothia numbering” refers to the numbering system set forth by Chothia et al., Journal of Molecular Biology, “Canonical Structures for the Hypervariable regions of immunoglobulins” (1987) and Chothia et al., Nature, “Conformations of Immunoglobulin Hypervariable Regions” (1989).

As used herein, the term “humanized antibody” refers to an antibody from a non-human species whose protein sequences have been modified to increase similarity to human antibodies. A humanized antibody may be produced by production of recombinant DNA coding for the CDRs of the non-human antibody surrounded by sequences that resemble a human antibody. In some embodiments, a humanized antibody is a chimeric antibody. In some embodiments, humanizing comprises insertion of the aforementioned CDRs into a human antibody scaffold or backbone. Humanized antibodies are well known in the art and any method of producing them that retains the aforementioned CDRs may be employed.

The term “monoclonal antibody” or “mAb” as used herein refers to an antibody obtained from a population of substantially homogeneous antibodies, i.e., the individual antibodies comprising the population are identical and/or bind the same epitope, except for possible variants that may arise during production of the monoclonal antibody, such variants generally being present in minor amounts. In contrast to polyclonal antibody preparations that typically include different antibodies directed against different determinants (epitopes), each monoclonal antibody is directed against a single determinant on the antigen. In addition to their specificity, the monoclonal antibodies are advantageous in that they are uncontaminated by other immunoglobulins. The modifier “monoclonal” indicates the character of the antibody as being obtained from a substantially homogeneous population of antibodies, and is not to be construed antibodies to be used in accordance with the methods provided herein may be made by the hybridoma method first described by Kohler et al, Nature 256:495 (1975), or may be made by recombinant DNA methods (see, e.g., U.S. Pat. No. 4,816,567). The “monoclonal antibodies” may also be isolated from phage antibody libraries using the techniques described in Clackson et al, Nature 352:624-628 (1991) and Marks et al, J. Mol. Biol. 222:581-597 (1991), for example.

A mAb may be of any immunoglobulin class including IgG, IgM, IgD, IgE or IgA. A hybridoma producing a mAb may be cultivated in vitro or in vivo. High titers of mAbs can be obtained in vivo production where cells from the individual hybridomas are injected intraperitoneally into pristine-primed Balb/c mice to produce ascites fluid containing high concentrations of the desired mAbs. mAbs of isotype IgM or IgG may be purified from such ascites fluids, or from culture supernatants, using column chromatography methods well known to those of skill in the art.

“Antibody fragments” comprise a portion of an intact antibody, preferably comprising the antigen binding region thereof. Examples of antibody fragments include Fab, Fab′, F(ab′)2, and Fv fragments; diabodies; tandem diabodies (taDb), linear antibodies (e.g., U.S. Pat. No. 5,641,870, Example 2; Zapata et al, Protein Eng. 8(10): 1057-1062 (1995)); one-armed antibodies, single variable domain antibodies, minibodies, single-chain antibody molecules; multi-specific antibodies formed from antibody fragments (e.g., including but not limited to, db-Fc, taDb-Fc, taDb-CH3, (scFv)4-Fc, di-scFv, bi-scFv, or tandem (di,tri)-scFv); and Bi-specific T-cell engagers (BiTEs).

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-binding sites and is still capable of cross-linking antigen.

“Fv” is the minimum antibody fragment that contains a complete antigen-recognition and antigen-binding site. This region consists of a dimer of one heavy chain and one light chain variable domain in tight, non-covalent association. It is in this configuration that the three surfaces of the VH-VL dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more Cysteines from the antibody hinge region. Fab′-SH is the designation herein for Fab′ in which the Cysteine residue(s) of the constant domains bear at least one free thiol group. F(ab′)2 antibody fragments originally were produced as pairs of Fab′ fragments that have hinge Cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains.

Depending on the amino acid sequence of the constant domain of their heavy chains, antibodies can be assigned to different classes. There are five major classes of intact antibodies: IgA, IgD, IgE, IgG, and IgM, and several of these may be further divided into subclasses (isotypes), e.g., IgG1, IgG2, IgG3, IgG4, IgA, and IgA2. The heavy chain constant domains that correspond to the different classes of antibodies are called a, delta, e, gamma, and micro, respectively. The subunit structures and three-dimensional configurations of different classes of immunoglobulins are well known.

“Single-chain Fv” or “scFv” antibody fragments comprise the VH and VL domains of antibody, wherein these domains are present in a single polypeptide chain. In some embodiments, the Fv polypeptide further comprises a polypeptide linker between the VH and VL domains that enables the scFv to form the desired structure for antigen binding. For a review of scFv see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy chain variable domain (VH) connected to a light chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies production is known in the art and is described in Natl. Acad. Sci. USA, 90:6444-6448 (1993).

The term “multi-specific antibody” is used in the broadest sense and specifically covers an antibody that has polyepitopic specificity. Such multi-specific antibodies include, but are not limited to, an antibody comprising a heavy chain variable domain (VH) and a light chain variable domain (VL), where the VHVL unit has polyepitopic specificity, antibodies having two or more VL and VH domains with each VHVL unit binding to a different epitope, antibodies having two or more single variable domains with each single variable domain binding to a different epitope, full length antibodies, antibody fragments such as Fab, Fv, dsFv, scFv, diabodies, bi-specific diabodies, triabodies, tri-functional antibodies, antibody fragments that have been linked covalently or non-covalently. “Polyepitopic specificity” refers to the ability to specifically bind to two or more different epitopes on the same or different target(s).

Monoclonal antibodies may be prepared using methods well known in the art. Examples include various techniques, such as those in Kohler, G. and Milstein, C, Nature 256: 495-497 (1975); Kozbor et al, Immunology Today 4: 72 (1983); Cole et al, pg. 77-96 in MONOCLONAL ANTIBODIES AND CANCER THERAPY, Alan R. Liss, Inc. (1985).

Besides the conventional method of raising antibodies in vivo, antibodies can be generated in vitro using phage display technology. Such a production of recombinant antibodies is much faster compared to conventional antibody production and they can be generated against an enormous number of antigens. Furthermore, when using the conventional method, many antigens prove to be non-immunogenic or extremely toxic, and therefore cannot be used to generate antibodies in animals. Moreover, affinity maturation (i.e., increasing the affinity and specificity) of recombinant antibodies is very simple and relatively fast. Finally, large numbers of different antibodies against a specific antigen can be generated in one selection procedure. To generate recombinant monoclonal antibodies, one can use various methods all based on display libraries to generate a large pool of antibodies with different antigen recognition sites. Such a library can be made in several ways: One can generate a synthetic repertoire by cloning synthetic CDR3 regions in a pool of heavy chain germline genes and thus generating a large antibody repertoire, from which recombinant antibody fragments with various specificities can be selected. One can use the lymphocyte pool of humans as starting material for the construction of an antibody library. It is possible to construct naive repertoires of human IgM antibodies and thus create a human library of large diversity. This method has been widely used successfully to select a large number of antibodies against different antigens. Protocols for bacteriophage library construction and selection of recombinant antibodies are provided in the well-known reference text Current Protocols in Immunology, Colligan et al (Eds.), John Wiley & Sons, Inc. (1992-2000), Chapter 17, Section 17.1.

An “antigen” is a molecule or a portion of a molecule capable of eliciting antibody formation and being bound by an antibody. An antigen may have one or more than one epitope. The specific reaction referred to above is meant to indicate that the antigen will react, in a highly selective manner, with its corresponding antibody and not with the multitude of other antibodies which may be evoked by other antigens.

In some embodiments, increasing the biological half-life or serum level of sCD28 is achieved by sCD28-based immunotherapy. In some embodiments, the sCD28-based immunotherapy comprises administering an agent of the invention to a subject in need thereof. In some embodiments, the sCD28-based immunotherapy comprises administering an anti-sCD28 antibody to a subject in need thereof.

As used herein, the term “immune response” refers to any response taken by the body to defend itself from pathogens or abnormalities. In one embodiment, an immune response comprises a response mediated or involving an immune cell.

In one embodiment, an immune response comprises any response activating or inhibiting the immune system or mediators of the immune system. In another embodiment, activation of an immune response comprises activation of an immune cell. In another embodiment, activation of an immune cell results in the proliferation of a sub-set of immune cells. In another embodiment, activation of an immune cell results in increased secretion of an immunologic mediator by the activated cell. In another embodiment, activation of an immune cell results in the engulfment and/or destruction of a pathogen, a foreign cell, a diseased cell, a molecule derived or secreted therefrom, or any combination thereof. In another embodiment, activation of an immune cell results in the engulfment and or destruction of a neighboring cell, such as, but not limited to, a cell infected by a virus. In another embodiment, activation of an immune cell results in the engulfment and/or destruction of a host cell, a molecule derived or secreted therefrom, or any combination thereof. In another embodiment, activation of an immune cell results in activating the secretion of antibodies directed to a certain molecule, epitope, pathogen, or any combination thereof.

In some embodiments, an immune response is a cytotoxic response. As used herein, cytotoxic response refers to a response comprising activation of the complement system, leading to cell lysis and/or other damage. In some embodiments, an immune response is a humoral response, i.e., involves production and secretion of antibodies. In some embodiments, an immune response is an innate response, i.e., involves the innate immune system. In some embodiments, an immune response is an acquired immune response, i.e., involves the acquired immune response.

In some embodiments, the subject is a graft recipient or a candidate for engraftment. In some embodiment, the graft comprises solitary cells, cell suspension, an organ, or any combination thereof. In some embodiments, the graft is an autologous graft. In some embodiment, the graft is a syngeneic graft. In some embodiments, the graft is an allogenic graft. In some embodiments, the graft is a xenograft. In some embodiments, the graft is a hematopoietic graft. In some embodiments, the graft comprises hematopoietic stem cells. In some embodiments, the graft is a non-hematopoietic graft.

In some embodiments, the subject is afflicted with sCD28-associated disease. As used herein, an “sCD28-associated disease” refers to any disease or condition which diverts from normal or proper homeostasis that is initiated by, promoted by, propagated by, involves sCD28, or any combination thereof. In some embodiments, sCD28 is used in diagnosing a sCD28-associated disease. In some embodiments, sCD28 is used in prognosis of a sCD28-associated disease. In some embodiments, levels of sCD28 correlate with prognosis of a sCD28-associated disease. In some embodiments, an sCD28-associated disease comprises sCD28 levels above 5 ng/ml.

In some embodiments, the subject's blood comprises elevated levels of sCD28. In some embodiments, the subject's blood does not comprise elevated levels of sCD28. In some embodiments, the subject's blood before the administering comprises elevated levels of sCD28. In some embodiments, the subject is afflicted with a disease or condition characterized by elevated levels of sCD28. In some embodiments, the levels are elevated above those of healthy subjects. In some embodiments, the subject's sCD28 levels are elevated by at least 5%, 10%, 15%, 20%, 25%, 30%, 40%, 50%, 60%, 70%, 80%, 90%, 100%, 150%, 200%, 250%, 300%, 350%, 400%, 500%, 600%, 700%, 800%, 900%, or 1000% above healthy subject levels. Each possibility represents a separate embodiment of the invention. In some embodiments, elevated levels are above 5, 6, 7, 8, 9, 10, 12, 14, 15, 16, 18, 20, 25, 30, 35, 40, 45 or 50 ng/ml of blood. Each possibility represents a separate embodiment of the invention. In some embodiments, the levels are elevated above 5 ng/ml. In some embodiments, the levels are elevated above 10 ng/ml. In some embodiments, the levels are elevated above 20 ng/ml. In some embodiments, the subject's blood comprises at least 5, 6, 7, 8, 9, 10, 12, 14, 15, 16, 18, 20, 25, 30, 35, 40, 45 or 50 ng sCD28 per ml of blood. Each possibility represents a separate embodiment of the invention. In some embodiments, the subject's blood prior to the administering comprises at least 5, 6, 7, 8, 9, 10, 12, 14, 15, 16, 18, 20, 25, 30, 35, 40, 45 or 50 ng sCD28 per ml of blood. Each possibility represents a separate embodiment of the invention. In some embodiments, the subject's blood comprises at least 5 ng/ml sCD28. In some embodiments, the subject's blood comprises at least 10 ng/ml sCD28. In some embodiments, the subject's blood comprises at least 20 ng/ml sCD28. In some embodiments, the subject's blood prior to the administering comprises at least 5 ng/ml sCD28. In some embodiments, the subject's blood prior to the administering comprises at least 10 ng/ml sCD28. In some embodiments, the subject's blood prior to the decreasing comprises at least 20 ng/ml sCD28.

In some embodiments, the subject is afflicted with allergy or an allergic reaction. In some embodiments, the allergic reaction results from an infectious disease or disorder. In some embodiments, the allergic reaction is a symptom of an infectious disease or disorder. In some embodiments, the allergic reaction is independent of an infectious disease or disorder. In some embodiment, the allergic reaction is stimulated in parallel to an infectious disease or disorder.

In some embodiments, the subject is afflicted with a cytokine release syndrome (CRS). As used herein, “cytokine release syndrome” refers to a systemic inflammatory response syndrome resulting from a complication of other disease or infection. In one embodiment, CRS is induced by or results from (e.g., an adverse effect) an immunotherapy, such as a monoclonal antibody drug. In one embodiment, CRS is induced by or results from an adoptive T-cell therapy. As used herein, the terms “CRS” and “cytokine storm” are interchangeable.

In some embodiments, the subject is afflicted with an infectious disease. Non-limiting examples for infectious disease, include, but are not limited to: urinary tract infection, gastrointestinal infection, enteritis, salmonellosis, diarrhea, nontuberculous mycobacterial infections, legionnaires' disease, hospital-acquired pneumonia, skin infection, cholera, septic shock, periodontitis, infection, sinusitis, bacteremia, neonatal infections, pneumonia, endocarditis, osteomyelitis, toxic shock syndrome, scalded skin syndrome, and food poisoning.

In some embodiments, the subject is afflicted with an autoimmune disease. As used herein, the term “autoimmune disease” refers to any disease or disorder resulting from an immune response against the subject's own tissue or tissue components (e.g., cells and molecules produced or secreted by same), or to antigens that are not intrinsically harmful to the subject. In some embodiments, the subject is afflicted with a T-cell-mediated autoimmune disease. Examples of an autoimmune disease include, but are not limited to Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile myositis (JM), Kawasaki disease, Lambert-Eaton syndrome, Leukocytoclastic vasculitis, Lichen planus, Lichen sclerosis, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Tolosa-Hunt syndrome (THS), Transverse myelitis, Type 1 diabetesmellitus, Ulcerative colitis (UC), Undifferentiated connective tissue disease (UCTD), Uveitis, Vasculitis, Vitiligo, and Vogt-Koyanagi-Harada Disease. In some embodiments, the autoimmune disease is selected from lupus erythematosus, asthma, Bechet's syndrome, Sjogren's syndrome, multiple sclerosis, autoimmune myasthenia gravis and neuromyelitis optica.

In some embodiments, the autoimmune disease is an autoimmune disease with elevated sCD28 levels. In some embodiments, the autoimmune disease comprises high sCD28 levels. In some embodiments, elevated and/or high sCD28 levels are levels at and/or above 5, 6, 7, 8, 9, 10, 12, 14, 15, 17, 20, 25, 30, 35, 40, 50, 60, 70, 80, 90 or 100 ng/ml. Each possibility represents a separate embodiment of the invention. In some embodiments, the autoimmune disease comprises high sCD28 levels. In some embodiments, elevated and/or high sCD28 levels are levels at and/or above 5, 10, 15, 20, 25, 30, 35, 40, 45, 50, 55, 60, 65, 70, 75, 80, 85, 90, 95, 100, 150, 200, 300, 400, 500, 600, 700, 800, 900, or 1000% of the levels in a healthy subject. Each possibility represents a separate embodiment of the invention. In some embodiments, the autoimmune disease does not comprise elevated levels of sCD28. In some embodiments, the autoimmune disease does not comprise high levels of sCD28. In some embodiments, high and/or elevated levels are as compared to a healthy subject.

In some embodiments, the subject has elevated sCD28 levels compared to a healthy subject. In some embodiments, the subject has non-elevated sCD28 levels compared to a healthy subject. In some embodiments, the subject and the healthy subject have comparable sCD28 levels. In some embodiments, a subject having non-elevated sCD28 levels or sCD28 levels comparable to a healthy subject, has 0 to less than 5% more sCD28 than a healthy subject. In some embodiments, a subject having non-elevated sCD28 levels or sCD28 levels comparable to a healthy subject, comprises less than 5 ng/ml of sCD28.

In some embodiments, a subject having elevated sCD28 levels comprises blood sCD28 levels elevated by at least 5%, 10%, 15%, 20%, 25%, 30%, 40%, 50%, 60%, 70%, 80%, 90%, 100%, 150%, 200%, 250%, 300%, 350%, 400%, 500%, 600%, 700%, 800%, 900%, or 1,000% above healthy subject levels, or any value and range there between. Each possibility represents a separate embodiment of the invention. In some embodiments, the blood sCD28 levels are elevated by 5-25%, 10-50%, 25-75%, 50-125%, 100-250%, 200-550%, 500-750%, or 700-1,000% above healthy subject levels. In some embodiments, a subject having elevated sCD28 levels comprises levels elevated above 5, 6, 7, 8, 9, 10, 12, 14, 15, 16, 18, 20, 25, 30, 35, 40, 45 or 50 ng/ml of blood. Each possibility represents a separate embodiment of the invention. In some embodiments, the levels are elevated above 5 ng/ml. In some embodiments, the levels are elevated above 10 ng/ml. In some embodiments, the subject's blood comprises at least 5, 6, 7, 8, 9, 10, 12, 14, 15, 16, 18, 20, 25, 30, 35, 40, 45 or 50 ng sCD28 per ml of blood. Each possibility represents a separate embodiment of the invention. In some embodiments, the subject's blood comprises at least 5 ng/ml sCD28. In some embodiments, the subject's blood comprises at least 10 ng/ml sCD28. In some embodiments, the subject's blood comprises more than 5, 6, 7, 8, 9, 10, 12, 14, 15, 16, 18, 20, 25, 30, 35, 40, 45 or 50 ng sCD28 per ml of blood. Each possibility represents a separate embodiment of the invention. In some embodiments, the subject's blood comprises more than 5 ng/ml sCD28. In some embodiments, the subject's blood comprises more than 10 ng/ml sCD28. In some embodiments, the subject's blood comprises more than 20 ng/ml sCD28.

According to the method of the invention, in some embodiments thereof, the administered agent increases the biological half-life or serum level of sCD28 in the subject, compared to the biological half-life without the administration. As used herein, “biological half-life” and “half-life” are synonymous and refer to the time it takes for half the amount of a compound to be removed from a cell, bodily fluid, or an organism, by any biological process, such as proteolysis, degradation, excretion, etc. In some embodiments, increasing the biological half-life comprises increasing the exposure of sCD28. In some embodiments, exposure comprises exposure time. In some embodiments, exposure is in plasma. In some embodiments, exposure is in serum. In some embodiments, exposure is in blood. In some embodiments, exposure is in a bodily fluid. The terms “biological half-life of sCD28” and “serum level of sCD28” are used herein interchangeably.

In some embodiments, according to the method of the invention, administering an agent having specific binding affinity to sCD28 to a subject results in increased sCD28 levels in the subject. In some embodiments, the increase is in the serum of the subject. In some embodiments, the increase is in the blood of the subject.

In some embodiments, the method comprises increasing sCD28 in the subject by at least 5%, 10%, 15%, 20%, 25%, 30%, 40%, 50%, 60%, 70%, 80%, 90%, 100%, 150%, 200%, 250%, 300%, 350%, 400%, 500%, 600%, 700%, 800%, 900%, or 1,000%, or any value and range therebetween. Each possibility represents a separate embodiment of the invention. In some embodiments, the method comprises increasing sCD28 in the subject by 5-25%, 10-50%, 25-75%, 50-125%, 100-250%, 200-550%, 500-750%, or 700-1,000%. Each possibility represents a separate embodiment of the invention.

As used herein, the term “subject” refers to any subject, particularly a mammalian subject, for whom therapy is desired, for example, a human.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

In some embodiments, the method comprises administering to the subject at least one agent having specific binding affinity to sCD28. As used herein, the terms “administering,” “administration,” and like terms refer to any method which, in sound medical practice, delivers a composition containing an active agent to a subject in such a manner as to provide a therapeutic effect. One aspect of the present subject matter provides for oral administration of a therapeutically effective amount of an agent to a patient in need thereof. Other suitable routes of administration can include parenteral, subcutaneous, intravenous, intramuscular, or intraperitoneal.

By another aspect, there is provided a pharmaceutical composition comprises an agent having specific binding affinity to sCD28 for use in suppressing an immune response.

By another aspect, there is provided a pharmaceutical composition comprises an agent having specific binding affinity to sCD28 for use in increasing the biological half-life or serum level of sCD28.

In some embodiments, the pharmaceutical composition comprises a therapeutically acceptable carrier, adjuvant or excipient. In some embodiments, the administering is administering the pharmaceutical composition.

As used herein, the term “carrier,” “excipient,” or “adjuvant” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelles, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

As used herein, the term “about” when combined with a value refers to plus and minus 10% of the reference value. For example, a length of about 1,000 nanometers (nm) refers to a length of 1,000±100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

Other terms as used herein are meant to be defined by their well-known meanings in the art.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Strategies for Protein Purification and Characterization—A Laboratory Course Manual” CSHL Press (1996); all of which are incorporated by reference. Other general references are provided throughout this document.

### Example 1

### Soluble CD28 Inhibits Activation of T Cells

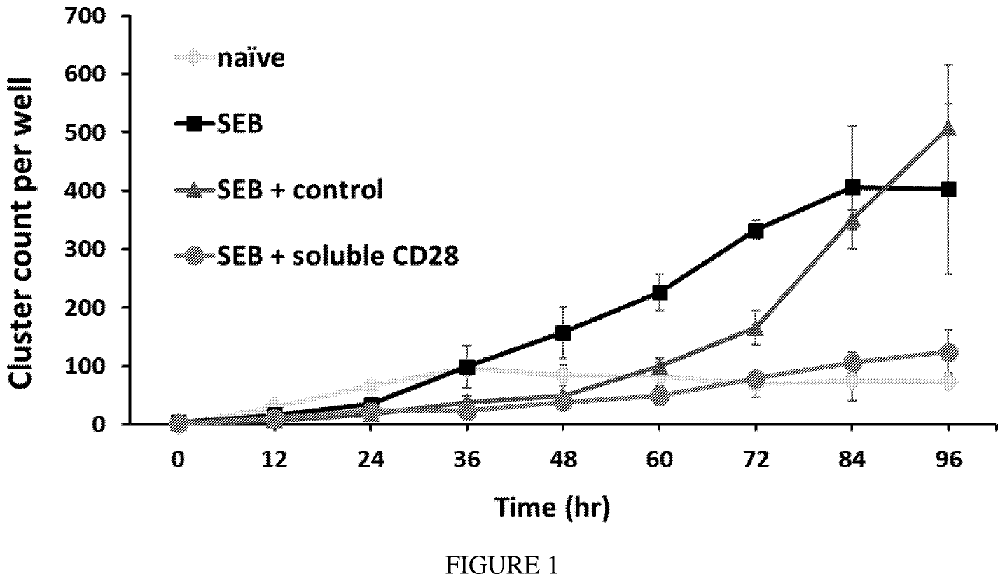

It is known that during in-vitro immune response antigen presenting cells (APC) cluster with one another and with other cell types, and clustering is essential for the antigen specific activation of resting lymphocytes. The inventors stimulated human PBMCs with SEB (1 ng/mL) in the presence of human IgG or recombinant human sCD28. Cluster formation was monitored by using the IncuCyte® S3 Live-Cell System by taking cellular images at the indicated times. Soluble CD28 was shown to diminish the amount and size of cluster formation during SEB immune response, and therefore was shown to inhibit the first steps of T cells specific activation by APCs (FIG. 1).

### Example 2

### Soluble CD28 Inhibits Effector Cytokines in a Dose Dependent Manner

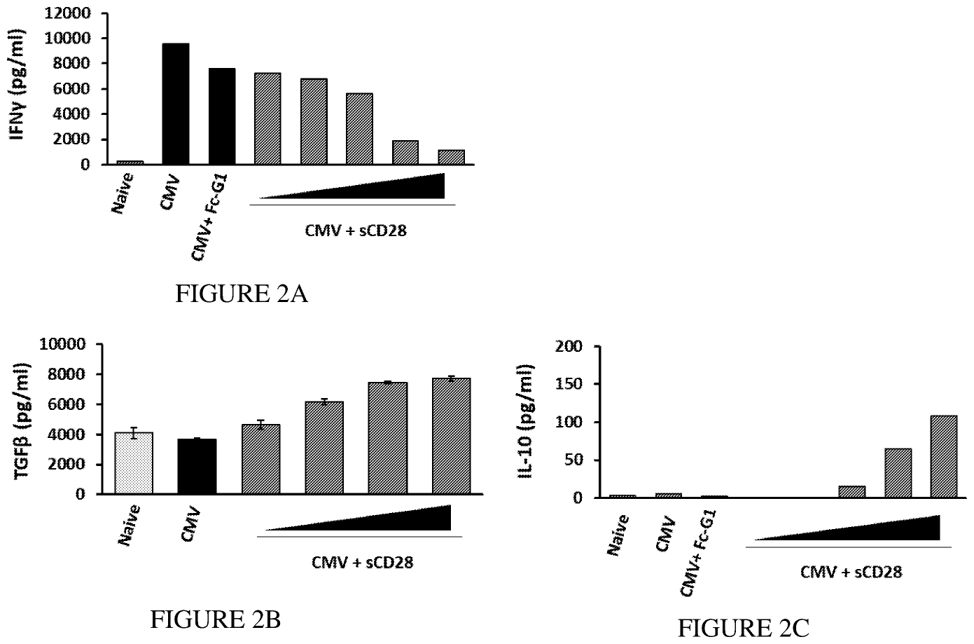

Isolated autologous monocytes and CD3 T cells were stimulated for 5 days with cytomegalovirus (CMV) peptide (0.5 μg/mL) with or without increasing concentrations of recombinant human sCD28. Naïve samples were not stimulated by CMV peptide. The concentrations of human: IFNγ, IL-10, and TGFβ in the supernatant were quantified with standardized sandwich ELISA (Biolegend). The sCD28 was shown to both inhibit effector cytokine secretion and promote secretion of immune-suppressive cytokines in monocytes mixed lymphocyte reaction (MLR) setting (FIG. 2A-C). Further to the above, the sCD28 activity, either inhibition of effector cytokines secretion or promotion of immune-suppressive cytokines secretion, was shown to be dose dependent. Therefore, increasing the amount of sCD28 provides the means for immunosuppressive therapy.

### Example 3

### Anti-sCD28 Antibody Increases Serum Exposure of sCD28

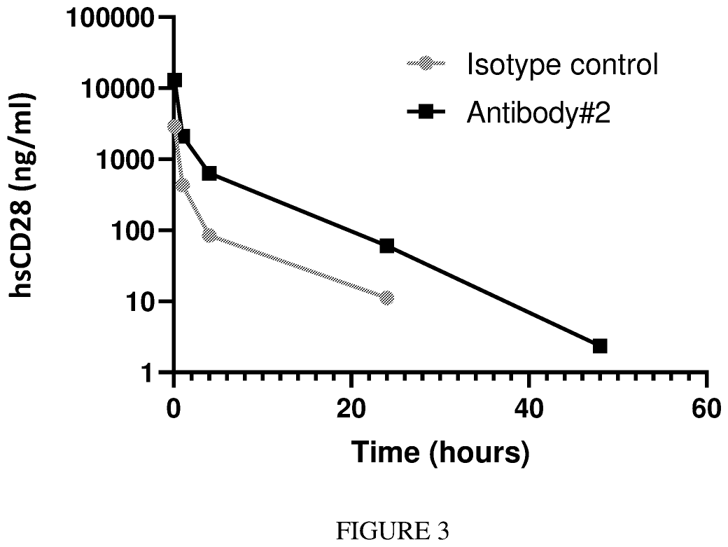

Elevated levels of sCD28 have been reported in several autoimmune settings (lupus erythematosus, asthma, Bechet's syndrome, Sjogren's syndrome, multiple sclerosis, autoimmune myasthenia gravis and neuromyelitis optica) however the clinical significance of these levels is still unclear. In light of the fact that sCD28 has now been clearly shown to have an immunosuppressing function it was investigated whether agents that bind the sCD28 might be able to increase its durability in serum and thus enhance its effects in an autoimmune setting. The effect of an anti-sCD28 antibody (i.e., antibody #2) on the total human sCD28 (hsCD28) plasma concentration was evaluated in a co-injection in-vivo model in normal mice. Recombinant human sCD28 was intravenously injected as a single dose of 0.5 mg/kg in the absence or presence of 5 mg/kg of antibody #2, and a time profile of the total hsCD28 plasma concentration was recorded (FIG. 3). Anti-sCD28 antibody was shown to increase the exposure of hsCD28 in the plasma.

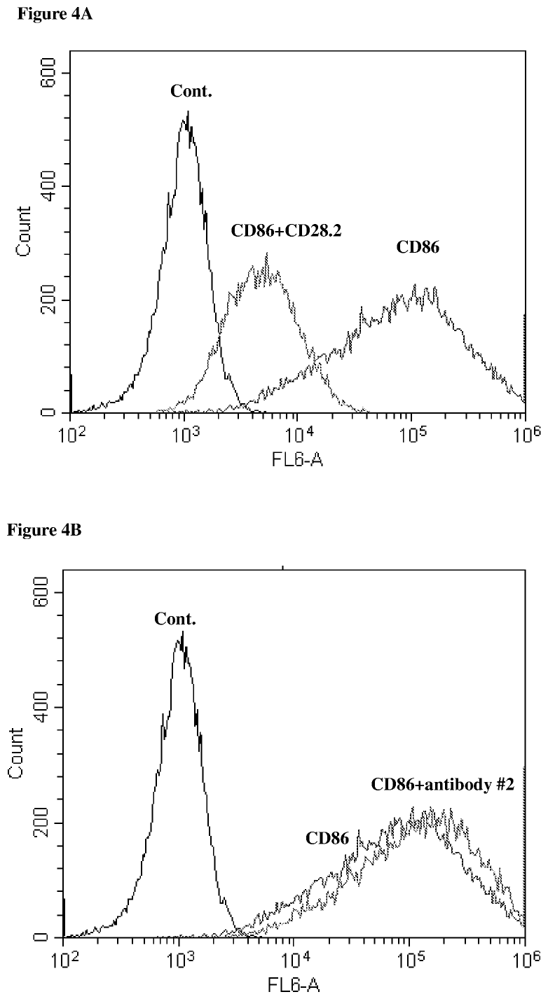

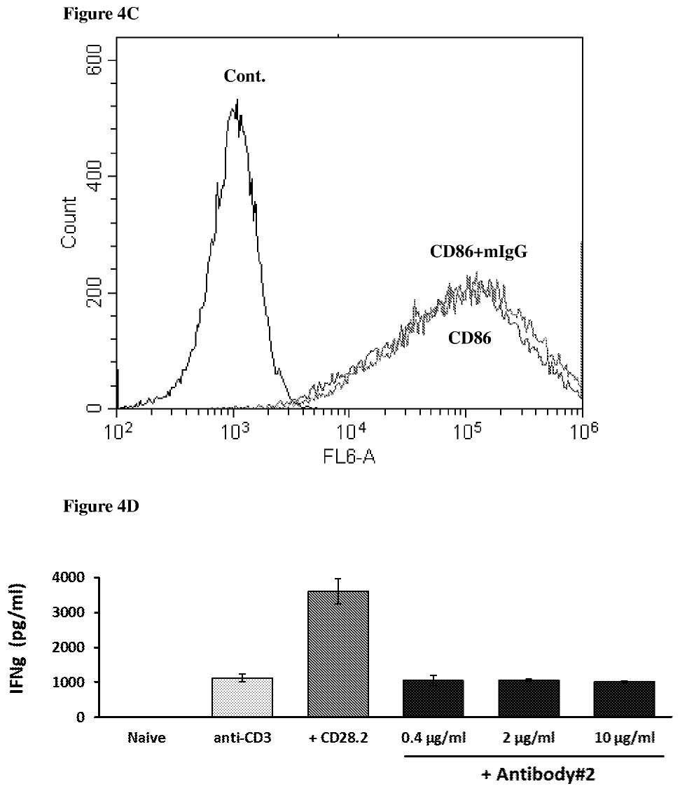

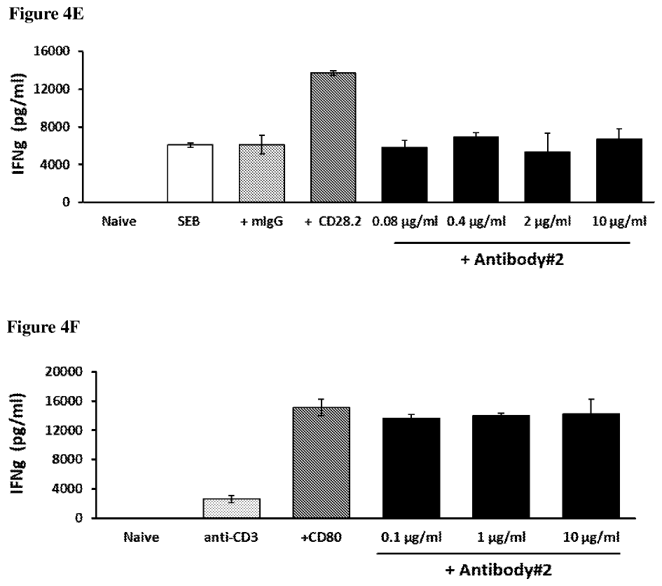

It was also determined that this antibody was neither a membranal CD28 (mCD28) agonist nor antagonist. Anti-CD28 antibody CD28.2 is known to stimulate T cell proliferation and cytokine secretion, as such it acts as a mCD28 agonist. Indeed, when binding of CD86 to mCD28 was measured by FACS, the addition of CD28.2 greatly decreased CD86 binding (FIG. 4A), indicating that CD28.2 binds to, or occludes, the ligand binding domain of mCD28. By contrast, antibody #2 (FIG. 4B) blocked binding of CD86 to mCD28, and indeed it's binding was comparable to the mIgG control (FIG. 4C).

Interferon gamma (IFNγ) secretion was measured as a representative of pro-inflammatory cytokine secretion by T cells. In the presence of anti-CD3 stimulation, antibody CD28.2 induced robust IFNγ secretion indicating that the T cells had been activated. By contrast, Antibody #2 all had no effect on IFNγ secretion at various concentrations (FIG. 4D). Thus, while CD28.2 acts as a mCD28 agonist, antibody #2 is not agonistic. Similar results were found when human PBMCs were stimulated with SEB (FIG. 4E).

Similarly, when human isolated T cells were stimulated with anti-CD3 antibodies, CD80-Fc behaves as an agonist increasing IFN gamma secretion. Addition of an antagonist should decrease the effect of CD80, however, when antibody #2 was added, no reduction in secretion was observed (FIG. 4F). This indicates that antibody #2 is also not antagonistic.

Thus, an agent having specific binding affinity to sCD28, which in turn increases the sCD28 serum exposure, and therefore prolongs or increases its immunosuppressive activity can be administered to a subject. It is also beneficial if the agent is neither an mCD28 agonist nor antagonist.

While certain features of the invention have been described herein, many modifications, substitutions, changes, and equivalents will now occur to those of ordinary skill in the art. It is, therefore, to be understood that the appended claims are intended to cover all such modifications and changes as fall within the true spirit of the invention.

1. A method for suppressing an immune response in a subject, comprising administering to said subject a therapeutically effective amount of an agent having specific binding affinity to soluble CD28 (sCD28), thereby suppressing an immune response in the subject.

2. The method of claim 1, wherein said agent increases the serum level of said sCD28 in said subject.

3. The method of claim 2, wherein said increase is at least a 20% increase as compared to the serum level without said administration.

4. The method of claim 1, wherein said agent is not a CD28 agonist.

5. The method of claim 1, wherein said agent is not a CD28 antagonist.

6. The method of claim 1, wherein said agent binds sCD28 with at least a 2-fold greater binding affinity compared to the binding affinity of said agent to membrane CD28 (mCD28).

7. The method of claim 1, wherein said agent does not bind mCD28.

8. The method of claim 1, wherein said sCD28 is in serum.

9. The method of claim 2, wherein said increasing the serum level of sCD28 comprises at least one of:
i. reducing sCD28 proteolysis;
ii. reducing sCD28 degradation;
iii. reducing sCD28 excretion;
iv. increasing sCD28 half-life; and
v. any combination thereof.

10. The method of claim 1, wherein said agent is an antibody or an antigen-binding portion thereof.

11. The method of claim 10, wherein said antibody or antigen-binding portion thereof comprises an IgG2 or IgG4 backbone.

12. The method of claim 10, wherein said antibody comprises three heavy chain CDRs (CDR-H) and three light chain CDRs (CDR-L), wherein:
CDR-H1 comprises the amino acid sequence set forth in SEQ ID NO: 1 (GYTLTNY), CDR-H2 comprises the amino acid sequence as set forth in SEQ ID NO: 2 (NTYTGK), CDR-H3 comprises the amino acid sequence as set forth in SEQ ID NO: 3 (GDANQQFAY), CDR-L1 comprises the amino acid sequence as set forth in SEQ ID NO: 4 (KASQDINSYLS), CDR-L2 comprises the amino acid sequence as set forth in SEQ ID NO: 5 (RANRLVD), and CDR-L3 comprises the amino acid sequence as set forth in SEQ ID NO: 6 (LQYDEFPPT);
CDR-H1 comprises the amino acid sequence set forth in SEQ ID NO: 7 (GYTFTSY), CDR-H2 comprises the amino acid sequence as set forth in SEQ ID NO: 8 (YPGDGD), CDR-H3 comprises the amino acid sequence as set forth in SEQ ID NO: 9 (NYRYSSFGY), CDR-L1 comprises the amino acid sequence as set forth in SEQ ID NO: 10 (KSSQSLLNSGNQKNYLT), CDR-L2 comprises the amino acid sequence as set forth in SEQ ID NO: 11 (WASTRES), and CDR-L3 comprises the amino acid sequence as set forth in SEQ ID NO: 12 (QSDYSYPLT); or
CDR-H1 comprises the amino acid sequence set forth in SEQ ID NO: 13 (GYTFTDY), CDR-H2 comprises the amino acid sequence as set forth in SEQ ID NO: 14 (NPNYDS), CDR-H3 comprises the amino acid sequence as set forth in SEQ ID NO: 15 (SSPYYDSNHFDY), CDR-L1 comprises the amino acid sequence as set forth in SEQ ID NO: 16 (SARSSINYMH), CDR-L2 comprises the amino acid sequence as set forth in SEQ ID NO: 17 (DTSKLAS), and CDR-L3 comprises the amino acid sequence as set forth in SEQ ID NO: 18 (HQRNSYPFT).

13. The method of claim 12, wherein said antibody or an antigen-binding portion thereof comprises a heavy chain comprising the amino acid sequence of SEQ ID NO: 19, 20, 21, 22, 23, or 24.

14. The method of claim 12, wherein said antibody or an antigen-binding portion thereof comprises a light chain comprising the amino acid sequence of SEQ ID NO: 25, 26, 27, 28, 29, or 30.

15. The method of claim 10, wherein said antibody or an antigen-binding portion thereof is selected from the group consisting of: a Fv, Fab, F(ab′)2, scFv or a scFv2 fragment.

16. The method of claim 10, wherein said antibody or an antigen-binding portion thereof is humanized.

17. The method of claim 1, wherein said subject is a graft recipient.

18. The method of claim 1, wherein said subject is afflicted with an autoimmune disease.

19. The method of claim 18, wherein said autoimmune disease is a sCD28-positive autoimmune disease.

20. The method of claim 18, wherein said autoimmune disease is selected from the group consisting of: lupus, rheumatoid arthritis, Crohn's disease, inflammatory bowel disease, Becht's disease, colitis, ulcerative colitis, diabetes, Graves' disease, and multiple sclerosis.

21. (canceled)

22. (canceled)


SEQUENCE LISTING (40 sequences)

SEQ ID NO: 1 - protein, 7 aa, Artificial Sequence
  Synthetic
GYTLTNY

SEQ ID NO: 2 - protein, 6 aa, Artificial Sequence
  Synthetic
NTYTGK

SEQ ID NO: 3 - protein, 9 aa, Artificial Sequence
  Synthetic
GDANQQFAY

SEQ ID NO: 4 - protein, 11 aa, Artificial Sequence
  Synthetic
KASQDINSYLS

SEQ ID NO: 5 - protein, 7 aa, Artificial Sequence
  Synthetic
RANRLVD

SEQ ID NO: 6 - protein, 9 aa, Artificial Sequence
  Synthetic
LQYDEFPPT

SEQ ID NO: 7 - protein, 7 aa, Artificial Sequence
  Synthetic
GYTFTSY

SEQ ID NO: 8 - protein, 6 aa, Artificial Sequence
  Synthetic
YPGDGD

SEQ ID NO: 9 - protein, 9 aa, Artificial Sequence
  Synthetic
NYRYSSFGY

SEQ ID NO: 10 - protein, 17 aa, Artificial Sequence
  Synthetic
KSSQSLLNSGNQKNYLT

SEQ ID NO: 11 - protein, 7 aa, Artificial Sequence
  Synthetic
WASTRES

SEQ ID NO: 12 - protein, 9 aa, Artificial Sequence
  Synthetic
QSDYSYPLT

SEQ ID NO: 13 - protein, 7 aa, Artificial Sequence
  Synthetic
GYTFTDY

SEQ ID NO: 14 - protein, 6 aa, Artificial Sequence
  Synthetic
NPNYDS

SEQ ID NO: 15 - protein, 12 aa, Artificial Sequence
  Synthetic
SSPYYDSNHFDY

SEQ ID NO: 16 - protein, 10 aa, Artificial Sequence
  Synthetic
SARSSINYMH

SEQ ID NO: 17 - protein, 7 aa, Artificial Sequence
  Synthetic
DTSKLAS

SEQ ID NO: 18 - protein, 9 aa, Artificial Sequence
  Synthetic
HQRNSYPFT

SEQ ID NO: 19 - protein, 117 aa, Artificial Sequence
  Synthetic
QIQLVQSGPELKKPGETVKISCKASGYTLTNYGMNWVKQAPGKGLKWMGWINTYTGKPTYVDDFKGRFAFSLETSASTAYLQINNLKNEDTATYFCARGDANQQFAYWGQGTLVTVS

SEQ ID NO: 20 - protein, 442 aa, Artificial Sequence
  Synthetic
QIQLVQSGPELKKPGETVKISCKASGYTLTNYGMNWVKQAPGKGLKWMGWINTYTGKPTYVDDFKGRFAFSLETSASTAYLQINNLKNEDTATYFCARGDANQQFAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 21 - protein, 118 aa, Artificial Sequence
  Synthetic
QVQLQQSGAELARPGASVKLSCKASGYTFTSYWMQWIKKRPGQGLEWIGAIYPGDGDTRYTQKFKGKATLTADKSSTTAYMQLSSLASEDSAVYFCARNYRYSSFGYWGQGTLVTVSA

SEQ ID NO: 22 - protein, 454 aa, Artificial Sequence
  Synthetic
QVQLQQSGAELARPGASVKLSCKASGYTFTSYWMQWIKKRPGQGLEWIGAIYPGDGDTRYTQKFKGKATLTADKSSTTAYMQLSSLASEDSAVYFCARNYRYSSFGYWGQGTLVTVSAAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 23 - protein, 121 aa, Artificial Sequence
  Synthetic
EVQLQQFGAELVKPGASVKISCKASGYTFTDYNMDWVKQSHGKSLEWIGDINPNYDSTAYNQKFMGKATLTVDKSSNTAYMELRSLTSEDTAVYYCARSSPYYDSNHFDYWGQGTSLTVSS

SEQ ID NO: 24 - protein, 445 aa, Artificial Sequence
  Synthetic
EVQLQQFGAELVKPGASVKISCKASGYTFTDYNMDWVKQSHGKSLEWIGDINPNYDSTAYNQKFMGKATLTVDKSSNTAYMELRSLTSEDTAVYYCARSSPYYDSNHFDYWGQGTSLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 25 - protein, 107 aa, Artificial Sequence
  Synthetic
DIKMTQSPSSMYASLGERVTITCKASQDINSYLSWFQQKPGKSPKTLIYRANRLVDGVPSRFSGSGSGQDYSLTISSLEYDDMGIYYCLQYDEFPPTFGAGTKLELK

SEQ ID NO: 26 - protein, 214 aa, Artificial Sequence
  Synthetic
DIKMTQSPSSMYASLGERVTITCKASQDINSYLSWFQQKPGKSPKTLIYRANRLVDGVPSRFSGSGSGQDYSLTISSLEYDDMGIYYCLQYDEFPPTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 27 - protein, 113 aa, Artificial Sequence
  Synthetic
DIVMTQSPSSLTVTAGEKVTLSCKSSQSLLNSGNQKNYLTWYQQKPGQPPQLLIYWASTRESGVPDRFTGSGSGTDFTLTISSVQAEDLAVYYCQSDYSYPLTFGAGTKLELK

SEQ ID NO: 28 - protein, 220 aa, Artificial Sequence
  Synthetic
DIVMTQSPSSLTVTAGEKVTLSCKSSQSLLNSGNQKNYLTWYQQKPGQPPQLLIYWASTRESGVPDRFTGSGSGTDFTLTISSVQAEDLAVYYCQSDYSYPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 29 - protein, 106 aa, Artificial Sequence
  Synthetic
QIVLTQSPAIMSASPGEKVTMTCSARSSINYMHWFQQKPGTSPKRWIYDTSKLASGVPARFSGSGSGTSYSLTISNMEAEDAATYYCHQRNSYPFTFGSGTKLEIK

SEQ ID NO: 30 - protein, 213 aa, Artificial Sequence
  Synthetic
QIVLTQSPAIMSASPGEKVTMTCSARSSINYMHWFQQKPGTSPKRWIYDTSKLASGVPARFSGSGSGTSYSLTISNMEAEDAATYYCHQRNSYPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 31 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 32 - protein, 202 aa, Homo sapiens
NKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 33 - protein, 27 aa, Homo sapiens
FWVLVVVGGVLACYSLLVTVAFIIFWV

SEQ ID NO: 34 - protein, 139 aa, Homo sapiens [sequence omitted]

SEQ ID NO: 35 - protein, 121 aa, Homo sapiens
NKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGEE

SEQ ID NO: 36 - protein, 16 aa, Homo sapiens
GKHLCPSPLFPGPSKP

SEQ ID NO: 37 - protein, 19 aa, Homo sapiens
HVKGKHLCPSPLFPGPSKP

SEQ ID NO: 38 - protein, 110 aa, Homo sapiens
MLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKG

SEQ ID NO: 39 - protein, 144 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSP

SEQ ID NO: 40 - protein, 126 aa, Homo sapiens
NKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSP